(19) United States
(12) Patent Application Publication
(10) Pub. No.: US 2026/0309710 A1
(43) Pub. Date: Oct. 8, 2026

(54) METHOD FOR CUSTOMIZING A BIOSENSOR TO MEASURE AN ANALYTE WITH HIGH PERFORMANCE PARAMETERS BY MEANS OF ARTIFICIAL INTELLIGENCE

(71) Applicant: digid GmbH, Mainz (DE)

(72) Inventors: Konstantin KLOPPSTECH, Mainz (DE); Nils KÖNNE, Mainz (DE); Constantin VON GERSDORFF, Mainz (DE)

(21) Appl. No.: 19/472,157

(22) PCT Filed: Apr. 5, 2024
(86) PCT No.: PCT/EP2024/059290

§ 371 Date: Oct. 3, 2025

(30) Foreign Application Priority Data
Apr. 5, 2023 (DE) 10 2023 108 795.9

(51) Int. Cl. G01N 33/569; G01N 27/04; G01N 33/53; G01N 33/543; G06N 20/00

(52) U.S. Cl. CPC G01N 33/56983; G01N 27/045; G01N 33/5308; G01N 33/5438; G06N 20/00; G01N 2469/10

(57) ABSTRACT

A method is provided for individualizing a biosensor for measuring a specific analyte. In an exemplary aspect, the method includes determining at least one quality parameter of the biosensor, determining at least one performance parameter and an associated threshold value of the biosensor based on the at least one quality parameter using a trained first artificial intelligence, and individualizing the biosensor for measuring a specific analyte by evaluating at least one of the performance parameters and the associated threshold value for an individual application scenario.

### TECHNICAL FIELD

The present invention relates to a method for individualizing a biosensor for measuring a specific analyte in order to derive an improved, preferably best possible qualitative and/or quantitative statement about the presence and/or a concentration of the analyte or analytes in the sample.

### PRIOR ART

A highly sensitive method for detecting and analyzing analytes in a sample consists in using an analyte-induced change in the surface tension of a miniaturized spring element, a so-called cantilever, in order to measure the presence and/or concentration of an analyte in a sample.

A sensor device for detecting an occurrence and/or a concentration and/or a quantity of an analyte in a sample, comprising a sensor, connection electronics and a housing, wherein the sensor is designed to convert chemical and/or biochemical information of an analyte in a sample into an electrical signal, wherein the sensor comprises a test lever which has a base and a deformable part, wherein a receptor layer for selectively receiving the analyte is applied at least on the deformable part, wherein the sensor comprises a reference lever which has a base and a deformable part, wherein a reference layer for selectively not receiving the analyte is applied on the deformable part, is known from WO 2022/200438 A1.

US 2022/0335817 A1 discloses a sensor device for detecting an environmental parameter and a method for determining information about a functional state of a sensor device.

### SUMMARY OF THE INVENTION

In order to further improve the accuracy of the detection of the analyte, a method for individualizing a biosensor for measuring a specific analyte having the features of claim 1 is proposed.

Accordingly, a method for individualizing a biosensor for measuring a specific analyte is proposed, which comprises the steps of determining at least one quality parameter of the biosensor, determining at least one performance parameter and an associated threshold value of the biosensor based on the at least one quality parameter using a trained first artificial intelligence, and individualizing the biosensor for measuring a specific analyte by evaluating at least one of the performance parameters and the associated threshold value for an individual application scenario.

In this way, a biosensor can be individualized during or after its production for use in a specific application scenario.

In the production of biosensors, for example in the production of MEMS biosensors by growing the MEMS structures onto a wafer, applying nanosensors or immobilizing biochemistry, different properties of the biosensors can occur as a result of statistical variations or fluctuations in the production process. Thus, some biosensors may be more suitable for a first application scenario, whereas other biosensors may be more suitable for a second application scenario.

By means of the proposed method for individualizing the biosensors, they can then be assigned precisely to an application scenario in accordance with their properties, in which the individualized biosensor has a good, preferably an optimal, measurement accuracy.

The quality parameter can be determined based on a property of the biosensor, wherein at least one electrical resistance is determined as the property, preferably all electrical resistances of a resistance bridge of the biosensor.

If the biosensor comprises at least one resistance bridge, at least one of the quality parameters can be a self-similarity of the electrical resistances of the resistance bridge.

It has emerged that the self-similarity of the resistances of the resistance bridge is a good indicator for the performance of the biosensor or of the biosensor and can therefore also be used as at least one of the quality parameters which can be used for individualizing the respective biosensor.

At least one of the performance parameters of the biosensor can comprise a sensitivity and/or a specificity and/or a yield. The performance of a biosensor can be described well for a specific analyte by means of these performance parameters.

It is of great importance for the user of a biosensor that the respective analysis results are consistent and reliable or the user receives an indication of the probability or the certainty with which the respective analysis results are affected.

In other words, the user wishes to know whether the test result obtained is reliable or not. However, the expected degree of reliability can vary depending on the field of application. In a virus test for humans, a very high level of reliability can be required in order, for example, to reliably order or cancel quarantine measures. In a home test on the virus for a preliminary test, whether one goes to the doctor, or also in the testing on an animal, for example a companion animal, the expected reliability can also be lower.

In order to quantify the reliability of tests, for example, the parameters of the sensitivity and of the specificity are known, by means of which a test can be classified. For example, in a test for the presence of a specific virus, these parameters can be specified as performance parameters in order to describe the reliability of the test.

The sensitivity of a test specifies which proportion of the tested persons is reliably recognized as sick (“positive”) by the test. The sensitivity is correspondingly defined as the number of persons tested as sick by the test divided by the number of persons actually sick in the group of persons tested with the test. In the case of a high sensitivity of the test, hardly a sick person is incorrectly evaluated as healthy, so that in a test with a high sensitivity a sick person is also recognized as sick with a high probability.

The specificity of a test specifies which proportion of the tested persons is reliably recognized as healthy (“negative”) by the test. The specificity is correspondingly defined as the number of persons tested as healthy by the test divided by the number of persons actually healthy in the group of persons tested with the test. In the case of a high specificity of the test, hardly a healthy person is incorrectly evaluated as sick, so that in a test with a high specificity a healthy person is also recognized as healthy with a high probability.

Correspondingly, these performance parameters are well suited for determining the performance of the biosensor.

The trained first artificial intelligence, for example a neural network, can determine at least one of the performance parameters and an associated threshold value based on a feature vector, wherein the feature vector is obtained from a data preparation of the at least one quality parameter.

The trained first artificial intelligence can be trained based on training data which are obtained with the following steps:
determining a time-resolved measurement curve of at least one specific analyte, as well as at least one sample without the specific analyte, each with a plurality of biosensors;
determining an individual measurement result and a validity parameter for each biosensor with a second artificial intelligence from the time-resolved measurement curve of the respective biosensor;
determining at least one quality parameter for each biosensor;
statistically evaluating the individual measurement results based on the associated validity parameters using threshold values, the associated quality parameters as well as the previously known result for each biosensor;
determining performance parameters from the statistical evaluation;
using the quality parameters, performance parameters and threshold values as training data.

The time-resolved measurement curves can preferably be recorded at one or different defined and thus known concentrations of the analyte. The training data can also be determined for different specific analytes, for example for different virus groups or bacteria.

Preferably, the measurements are recorded with at least one defined measurement routine, for example the measurement routine which is also used in the later use of the respective type of biosensors. Different measurement routines, which can differ depending on the application scenario or depending on the measuring apparatus used, can also be used for the measurement.

The time-resolved measurement curves can additionally also be recorded at at least one defined disturbance of the measurement, such as for example a premature termination of the measurement, an excessive temperature change during the measurement, a displacement of the biosensor during the measurement. Training data for different disturbances, such as for example disturbances due to temperature or interfering proteins, can also be generated by a corresponding adaptation of the samples and/or measurement routine.

In this way, optimized training data can be obtained in order to have a representative data set for training a first trained artificial intelligence, so that in turn the trained first artificial intelligence can carry out a precise individualization of a biosensor.

In particular, the trained first artificial intelligence can be trained based on training data which comprise the self-similarity of the electrical resistances of a resistance bridge of each biosensor as quality parameters.

The trained first artificial intelligence can be trained based on training data which comprise the sensitivity and/or the specificity and/or the yield of each biosensor as performance parameters.

By training the trained first artificial intelligence, a particularly good prediction of the suitability of the individual biosensor for a specific application scenario can be made, so that an individualization of the biosensor is reliably possible and the measurement of the analyte can be reliably achieved or the economically best application purpose of the biosensor can be determined.

The biosensor can run through no or at least one further production step based on its individualization, wherein the no or at least one further production step preferably comprises a storage of quality parameters and/or performance parameters and/or threshold values on the biosensor or a database and/or the at least one further production step comprises a functionalization of the biosensor, wherein the functionalization of the biosensor preferably comprises an application of a receptor layer and/or reference layer.

The individualization of biosensors makes it possible to assign biosensors early in the production or at any other time to at least one suitable application scenario. Furthermore, a biosensor whose individualization does not produce a suitable application scenario and/or whose performance parameters are insufficient or inconsistent can be sorted out early and/or correspondingly post-processed. In this way, production resources and/or material can be saved. And it can be ensured that each biosensor which has been assigned to an application scenario in the individualization method is also suitable for this application scenario, preferably also best possible.

In addition, the individualization of biosensors which are temporarily stored, for example, enables a targeted and speedy continuation and/or reintegration of the individual biosensors into their production process. The individualization of the biosensors enables a determination of an adequate application scenario for each of the biosensors. Subsequently, each of the biosensors can thus possibly run through at least one further production step corresponding to the individual application scenario.

Data from the individualization of the biosensors, such as, for example, their performance parameters, application scenario and/or threshold value, can also be stored in a memory or can also be stored in the memory before the temporary storage and can be used for the continuation and/or reintegration of the individual biosensors into their production process. Thus, the individualization of biosensors enables a targeted and efficient control of the production and logistics of biosensors, comprising their inventory. A management system for production and logistics which can be used, inter alia, for the production of biosensors can thus comprise and use the method for the individualization of biosensors. Biosensors can also be checked again for their performance after a transport, if appropriate on the basis of a preceding individualization.

Consequently, biosensors which have been damaged due to transport damage during the transfer can then be sorted out and/or post-processed by the check.

The method for the individualization of biosensors is also suitable as a tool for the reuse of already used biosensors for measuring a specific analyte. On the basis of an application of the method for the individualization of the already used biosensor, its performance is determined after use. Thus, in the event that the performance of the already used biosensor is still sufficiently ensured, the already used biosensor can run through at least one further production step or neutralization step in order to be subsequently used again for a measurement of a specific analyte.

Furthermore, a method for generating training data for training a trained first artificial intelligence for use in the individualization of biosensors, comprising the following steps is proposed:
determining a time-resolved measurement curve of at least one specific analyte, as well as at least one sample without the specific analyte, each with a plurality of biosensors;
determining an individual measurement result and a validity parameter for each biosensor with a second artificial intelligence from the time-resolved measurement curve of the respective biosensor;
   determining at least one quality parameter for each biosensor;
   statistically evaluating the individual measurement results based on the associated validity parameters using threshold values, the associated quality parameters as well as the previously known result for each biosensor;
   determining performance parameters from the statistical evaluation;
   using the quality parameters, performance parameters and threshold values as training data.

The time-resolved measurement curves can preferably be recorded at one or different defined and thus known concentrations of the analyte. The training data can also be determined for different specific analytes, for example for different virus groups or bacteria.

Preferably, the measurements are recorded with at least one defined measurement routine, for example the measurement routine which is also used in the later use of the respective type of biosensors. Different measurement routines, which can differ depending on the application scenario or depending on the measuring apparatus used, can also be used for the measurement.

The time-resolved measurement curves can additionally also be recorded at at least one defined disturbance of the measurement, such as for example a premature termination of the measurement, an excessive temperature change during the measurement, a displacement of the biosensor during the measurement. Training data for different disturbances, such as for example disturbances due to temperature or interfering proteins, can also be generated by a corresponding adaptation of the samples and/or measurement routine.

In this way, optimized training data can be obtained in order to have a representative data set for training a first trained artificial intelligence, so that in turn the trained first artificial intelligence can carry out a precise individualization of a biosensor.

These specific training data subsequently allow a training of a trained first artificial intelligence, so that an individualization of biosensors can be reliably carried out.

The quality parameter determined for each biosensor can comprise the self-similarity of the resistances in a resistance bridge of each biosensor.

Furthermore, a biosensor for measuring a specific analyte is proposed, comprising at least one resistance bridge, wherein the biosensor is individualized for a specific application scenario for measuring the specific analyte based on a quality parameter determined from the resistance bridge.

The quality parameter can be the self-similarity of the resistances of the resistance bridge.

The biosensor envisaged here can be a biosensor for converting chemical and/or biochemical information of an analyte into an electrical signal in a sample. The biosensor can comprise a test cantilever which has a base and a deformable part, wherein a receptor layer for selectively receiving the analyte is applied at least on the deformable part, and a reference cantilever which has a base and a deformable part, wherein a reference layer for selectively not receiving the analyte is applied on the deformable part.

The base of the test cantilever and/or the base of the reference cantilever can be designed as a rigid base. A rigid base is understood here to mean that a deformation of the respective cantilever, that is to say of the test cantilever and/or of the reference cantilever, with respect to the deformable part of the respective cantilever does not take place or substantially does not take place. The rigid base is connected here, for example, to a substrate, supported by a substrate or machined out of the substrate. The deformable part of the test cantilever and/or of the reference cantilever, by contrast, is not supported by the substrate, but rather projects beyond an edge of the substrate and is designed correspondingly freely.

The deformable part of the test cantilever and/or of the reference cantilever can be designed to be deflectable, for example. Here, a deflection of the respective cantilever can be achieved, for example, about a bending edge formed in a transition region between base and deflectable region. The bending edge is here, for example, the edge of the substrate along which the cantilever is divided into the base and the deformable part.

The deformation of the respective cantilever in its deformable part is, however, not limited to a raising or lowering deformation; the cantilever can also be deformed in itself, for example arched or wavy or distorted.

A sample refers here to a limited quantity of a substance which has been taken from a larger quantity of the substance, for instance from a reservoir, wherein the composition of the sample is representative of the composition of the substance in the reservoir and accordingly the corresponding occurrence in the reservoir can be inferred from the substance occurrence and substance compositions of the sample.

For example, a sample can be a saliva sample, or a blood sample, or lymph, or urine, or sweat, or intertissue fluid, or a swab, in particular a throat swab or a nasal swab or a sinus swab, or tissue taken. A sample comprises in particular any type of biological sample, that is to say in particular also samples of animals.

A sample can also be a non-biological sample, for example a sample of a chemical substance. A gaseous sample is also possible.

An analyte is here the substance whose presence in the sample is to be qualitatively and/or quantitatively detected or is to be detected with the biosensor. The analyte can in particular be present directly in the sample, or be dissolved in the sample or adhere to the sample or to a part of the sample, in particular a sample particle. The analyte can also enter into a chemical, biological and/or physical interaction with the sample, with the result that the analyte can only be detected indirectly via a corresponding interaction.

In particular, a sample form can be converted into a further sample form, with the result that the analyte, or the occurrence thereof, can be detected in a simple and reliable manner. For example, a swab can be dissolved in a liquid, with the result that the swab dissolved in the liquid is then the actual sample which is examined for the analyte.

The analyte in the sample can also be chemically pretreated, for example by—if the analyte is a virus-disrupting the virus envelope in order to reach nucleocapsid antigens. Furthermore, the analyte can also be “labeled” by such a pretreatment in order to amplify the measurement signal. For this purpose, for example conjugated antibodies can bind to antigens of the analyte in order to generate as great a deformation as possible on the cantilever system.

The sample then contains the chemical information and/or biochemical information about the analyte. The chemical information can comprise, for example, the type of analyte, the concentration of the analyte, the occurrence of the analyte, the weight of the analyte, the reactivity of the analyte, the density of the analyte, etc. The biochemical information comprises the same properties as the chemical information, but these substances can arise, for example, as a result of biological processes. In particular, the term biochemical information is used when the analyte has a particular influence on the biological circulation, for example the metabolism, or on the immune system.

The test cantilever comprises a passive and an active test transducer and the reference cantilever comprises a passive and an active reference transducer.

The chemical and/or biochemical information is converted into an electrical signal. This can mean that an electrical signal can be changed or built up from the chemical composition of the analyte.

This can relate, for example, to the conductivity of a circuit. For example, a first piece of biochemical information can be present when the circuit conducts, and a second piece of biochemical information can be present when the circuit does not conduct or has a reduced conductivity.

In addition to the direct accessibility of the information, such as, for example, via the conductivity, however, it is also possible to infer the biochemical information via a physical and/or chemical process and/or an interaction.

For this purpose, the proposed biosensor comprises a reference cantilever and a test cantilever. A cantilever is here a spring element which has a base and a deformable part. The base is accordingly an immovable part of the cantilever which is arranged, in particular, in a stationary manner on a substrate. The deformable part of the cantilever is arranged on the base and projects beyond the base.

In particular, the base and the cantilever can be formed integrally with one another. In other words, the deformable part of the cantilever is suspended on one side on the base. In that the deformable part projects beyond the substrate, the deformable part of the cantilever can be bent, deflected and stretched. The spatial boundary beyond which the cantilever is bendable or the cantilever transitions from the base into the deformable part is called a bending edge. The bending edge is usually an edge of the substrate when the cantilever projects beyond the base.

If the cantilever is bent, material stresses and forces which can be measured emerge in or on the material of the cantilever. If such a material stress and/or force can be measured, a bending of the cantilever can be inferred therefrom.

The transducers have the purpose of determining or measuring the deformation of the cantilever. The active transducers are arranged on the deformable parts of the cantilever, whereas the passive transducers are arranged on the bases, for example the bases, of the cantilever. In particular, electrical properties of a circuit can be influenced via the transducers.

For example, a deformation of the cantilever can lead to the resistance of a transducer, for example of an active transducer, increasing, while no deformation of the cantilever also causes no change in the resistance of the transducer. This can take place, for example, via a design of the transducers according to the principle of a strain gauge, as a result of which a deformation of the respective cantilever manifests itself in a change in length of the strain gauge of the transducer applied thereto and therefore a deformation of the cantilever can be detected directly by a change in the resistance of the strain gauge.

Therefore, the chemical and/or biochemical information of the analyte can be detected via a deformation of the cantilever, a subsequent registration via a transducer, and finally via a change in an electrical property of a circuit.

The deformation of the cantilever can be brought about in a substance-specific manner by a suitable coating. For this reason, the reference cantilever has a reference layer for selectively not receiving the analyte, while the test cantilever has a receptor layer for receiving the analyte.

A receptor layer is here a substance which can interact with the analyte. This in turn means that the receptor layer is selected specifically for each analyte. Analogously, a reference layer is a substance which cannot interact with the analyte. The reference layer is therefore also selected specifically for the analyte.

In this case, interaction means that the analyte is in chemical and/or biochemical and/or physical interaction with the receptor layer. In particular, the interaction can consist in binding of the analyte to the receptor layer. An interaction can furthermore consist in absorption or adsorption or non-specific adhesion of the analyte to the receptor layer.

The receptor and reference layers are preferably chemically identical with respect to possible interference influences and preferably differ only by the interaction with the analyte. A substance which is not the analyte accordingly interacts just as strongly or just as weakly with the receptor layer as with the reference layer.

The selective reception of the analyte on the test cantilever has the effect that a force acts on the test cantilever by the analyte, with the result that the test cantilever reacts sensitively to the analyte. Accordingly, the other substances of the sample which are not the analyte contribute only to a background noise in the form of a background bending on the test cantilever. The force on the test cantilever increases, for example, the faster the greater the concentration of the analyte in the sample or the faster the surface of the cantilever is covered with the analyte. A maximum force possible for the respective design is achieved with a complete covering of the cantilever.

The selective non-reception of the analyte on the reference cantilever, by contrast, has the effect that no force acts on the reference cantilever by the analyte, with the result that only the substances which are not the analyte contribute to a background noise in the form of a background bending of the reference cantilever.

This acting force can bring about a deformation in the case of the deformable part of the test cantilever, while the deformable part of the reference cantilever is not bent. The basis for the deflection of the cantilever is the change in the surface tension as a result of the interaction with the analyte. The change in the surface tension leads to an expansion or compression of the upper (or lower) surface of the cantilever. The different expansion or compression on the upper side and underside brings about an internal force or material stress in the material, which internal force or material stress leads to the deformation.

In the case of the proposed biosensor, the measuring method is drastically simplified by the selective non-reception of the analyte by the reference cantilever, since the reference cantilever is not sensitive to the analyte and therefore the analyte also does not contribute to the background noise. Only the substances which are not the analyte contribute here to the background noise of the reference cantilever. To a certain extent, the selective non-reception of the analyte on the reference cantilever can have the effect that the reference cantilever is exposed to the same turbulences, the same thermal drift and the same influence of all substances which are not the analyte, as in a reference sample. However, with the difference that the reference signal is determined directly in the sample liquid, or if appropriate in the case of a gaseous sample at the corresponding point.

In particular, a reference cantilever with a reference layer and a test cantilever with a receptor layer bring about a significantly more specific analysis of the analyte than only a reference cantilever without a receptor layer, since both the reference layer and the receptor layer have a specific interaction or non-interaction with the analyte.

The construction of the biosensor with reference cantilever and test cantilever has the advantage that two measurements can be carried out simultaneously in the sample, wherein the measurement of the reference cantilever can calibrate the measurement of the test cantilever. Environmental influences, for instance chemical, thermal, mechanical, electrical and fluidic interference influences, on the respective measurement can thereby be reduced, with the result that an occurrence of the analyte can be inferred from the comparison of the measurement on the test cantilever and on the reference cantilever.

These forces or material stresses, for example expansions or compressions, which act on the cantilever can finally be detected by the transducers, wherein differently strong stresses are detected by the transducers by differently strong expansions or compressions.

### BRIEF DESCRIPTION OF THE FIGURES

Preferred further embodiments of the invention are explained in more detail by the following description of the figures.

In the figures:

FIG. 1 shows a first schematic embodiment of an exemplary biosensor for converting chemical and/or biochemical information;

FIG. 2 shows a further schematic embodiment of a biosensor;

FIG. 3 shows a schematic possibility of connecting the electrodes of a biosensor in the form of a resistance bridge;

FIG. 4 shows a schematic flowchart of a method for individualizing an individual, produced biosensor for measuring a specific analyte;

FIG. 5A shows a schematic exemplary embodiment for determining voltages and/or electrical resistances at a resistance bridge of a biosensor with a measuring device, here in the form of a schematically shown multimeter;

FIG. 5B shows an exemplary embodiment for determining the at least one property of the biosensor with a schematically illustrated camera system;

FIG. 6A shows schematically illustrated neural network as an exemplary embodiment for a trained first artificial intelligence for determining performance parameters and an associated threshold value;

FIG. 6B shows a schematic flowchart comprising a second artificial intelligence for generating training data;

FIG. 7 shows a schematic representation of a training data set comprising the performance parameters sensitivity, specificity and yield depending on the self-similarity of a resistance bridge and a threshold value;

FIG. 8 shows a schematic flowchart as an exemplary embodiment for evaluating a measurement of a biosensor on the basis of its individualization by means of a second artificial intelligence; and

FIG. 9 shows a schematic flowchart of an exemplary embodiment for running through no or at least one further production step of a biosensor on the basis of its individualization.

### DETAILED DESCRIPTION OF PREFERRED EXEMPLARY EMBODIMENTS

Preferred exemplary embodiments are described below with reference to the figures. In this case, identical, similar or identically acting elements are provided with identical reference signs in the different figures, and repeated description of these elements is partly dispensed with in order to avoid redundancies.

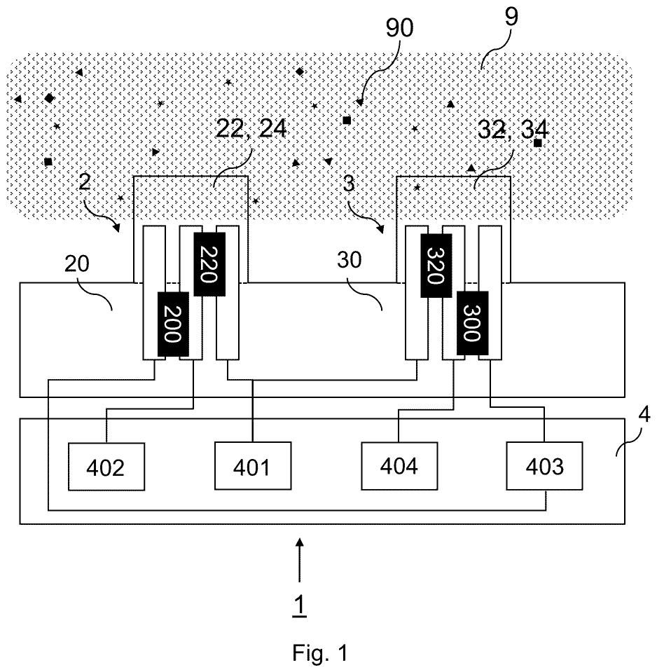

FIG. 1 schematically shows a first embodiment of an exemplary biosensor 1 for converting chemical and/or biochemical information. The biosensor 1 comprises a test cantilever 2 which in turn has a base 20 and a deformable part 22. A passive test transducer 200 is arranged on the base 20, while an active test transducer 220 is arranged on the deformable part 22.

Analogously to the test cantilever 2, the biosensor 1 also has a reference cantilever 3 which in turn has a base 30 with a passive reference transducer 300, and a deformable part 32 which has an active reference transducer 320.

The transducers have the purpose of determining or measuring the deformation of the cantilever. The active transducers are arranged on the deformable parts of the cantilever, whereas the passive transducers are arranged on the bases, for example the bases, of the cantilever. In particular, electrical properties of a circuit can be influenced via the transducers.

For example, a deformation of the cantilever can lead to the resistance of a transducer, for example of an active transducer, increasing, while no deformation of the cantilever also causes no change in the resistance of the transducer. This can take place, for example, via a design of the transducers according to the principle of a strain gauge, as a result of which a deformation of the respective cantilever manifests itself in a change in length of the strain gauge of the transducer applied thereto and therefore a deformation of the cantilever can be detected directly by a change in the resistance of the strain gauge.

Therefore, the chemical and/or biochemical information of the analyte can be detected via a deformation of the cantilever, a subsequent registration via a transducer, and finally via a change in an electrical property of a circuit.

The transducer can also detect a contraction of the surface on which it is arranged. However, in the embodiments shown, the transducers are always arranged on surfaces at which an expansion is expected.

However, the expansion and/or change in the surface tension and/or force which the transducer detects can also be a bending force or a shearing force or can be caused by a bending force or shearing force or can generally be based on the modulus of elasticity of the respective cantilever. In particular, as a result of the fastening of the deformable part 22, 32 to the base 20, 30, the deformable part 22, 32 is oriented along a bending curve on account of a force effect which is oriented by a change in the surface tension of the test cantilever. The resulting bending curve is given, in particular, by the geometry, in particular the area moment of inertia of the cantilever, and by the mass of the cantilever and the modulus of elasticity. The bending curves can be described, for example, according to bar theory.

The transducers 200, 220, 300, 320 are each connected to electronics 4 which are capable of recording or forwarding a measurement signal of the transducers 200, 220, 300, 320, while the electronics 4 are likewise capable of supplying the transducers 200, 220, 300, 320 with current and/or voltage.

The transducers 300, 320, 200, 220 are preferably electrically connected via electrodes 401, 402, 403, 404. In particular, the active test transducer 220 is connected to the active reference transducer 320 via the electrode 401. Furthermore, the passive test transducer 200 is connected to the passive reference transducer 300 via the electrode 403. The active test transducer 220 is additionally connected to the passive test transducer 200 via the electrode 402, whereas the active reference transducer 320 is connected to the passive reference transducer 300 via the electrode 404. This results in a total of four electrodes via which the transducers are electrically connected to one another.

In this case, electrical connection can be achieved, in particular, by the transducers being applied to electrodes which are already present, with the result that a conductive connection arises.

The biosensor 1 has the task of indicating the occurrence and/or the concentration and/or the quantity of an analyte 90 in a sample 9.

In FIG. 1, the sample 9 is a liquid which has been produced, for example, by carrying out a swab, in particular a nasal swab or a throat swab of a test person. It is also conceivable for the sample to comprise a liquid on an object, such as, for example, a rope or a cotton wadding. However, it may also be the case that the sample 9 is saliva or blood or another body fluid. However, it may also be the case that the sample 9 is a gargle liquid with which the test person has gargled. It may also be the case that the sample 9 has been obtained and/or synthesized from a tissue sample or from another substance taken from the test person. The analyte 90 can here be dissolved in the sample, or be present in an undissolved manner as a suspension or dispersion or emulsion.

However, the sample 9 and the analyte 90 of interest may also be of non-biological origin.

In any case, the biosensor 1 is intended to examine the sample 9 with regard to the occurrence and/or a concentration and/or a quantity of the analyte 90 in the sample 9. For this purpose, a receptor layer 24 with which an analyte 90 can interact is applied on the test cantilever 2. By way of example, the receptor layer 24 can adsorb or absorb the analyte 90. During adsorption, the analyte 90 would adhere to the surface of the receptor layer 24, whereas during absorption the analyte 90 would penetrate the interior of the receptor layer 24.

If the sample 9 has a specific analyte 90, it can thus interact with the receptor layer 24. This can lead to the surface tension of that section of the deformable part 22 of the test cantilever 2 which is covered with the receptor layer 24 changing. This change in the surface tension can be registered by the active test transducer 220, which is in turn interpreted as a measurement signal in the electronics 4.

The change in the surface tension of that section of the deformable part 22 of the test cantilever 2 which is covered with the receptor layer 24 can also lead to a deformation of the deformable part 22 of the test cantilever 2. The active test transducer 220 can therefore also register a deformation of the deformable part of the test cantilever 2, which is in turn interpreted as a measurement signal in the electronics 4.

However, a force effect can already be registered by the active test transducer 220 on account of the interaction with the sample liquid of the sample 9, for example in which only a surface tension of the liquid acts on the deformable part 22 of the test cantilever 2. Accordingly, in this case, the presence of an analyte 90 is not responsible for a correspondingly resulting deformation or change in the surface tension.

In order to determine the magnitude of this basic effect of the sample 9 on the test cantilever 2, the reference cantilever 3 is also brought into contact with the sample 9 simultaneously with the test cantilever 2. For this purpose, the reference cantilever 3 has a reference layer 34 with which an analyte 90 cannot interact or a reference layer 34 which cannot adsorb or absorb the analyte 90.

Here, an interaction with the analyte 90 is to be avoided in order to enable a differentiation with respect to the measurement signal of the test cantilever 2.

By both the test cantilever 2 and the reference cantilever 3 interacting with the sample 9, both cantilevers 2, 3 interact with the sample 9 in a similar manner. Here, however, the difference is that the test cantilever 2 can additionally interact with a possibly present analyte 90 via its reference layer 24. Accordingly, the measurement signals of the active transducers 220, 320 differ if an analyte 90 occurs in the sample 9. Accordingly, in the simplest case, the quantity of the occurrence of the analyte 90 in the sample 9 can be inferred via the magnitude of the difference in the measurement signals.

The deformation of the cantilever can be brought about in a substance-specific manner by a suitable coating. For this reason, the reference cantilever has a reference layer for selectively not receiving the analyte, while the test cantilever has a receptor layer for receiving the analyte.

A receptor layer is here a substance which can interact with the analyte. This in turn means that the receptor layer is selected specifically for each analyte. Analogously, a reference layer is a substance which cannot interact with the analyte. The reference layer is therefore also selected specifically for the analyte.

In this case, interaction means that the analyte is in chemical and/or biochemical and/or physical interaction with the receptor layer. In particular, the interaction can consist in binding of the analyte to the receptor layer. An interaction can furthermore consist in absorption or adsorption or non-specific adhesion of the analyte to the receptor layer.

The receptor and reference layers are preferably chemically identical with respect to possible interference influences and preferably differ only by the interaction with the analyte. A substance which is not the analyte accordingly interacts just as strongly or just as weakly with the receptor layer as with the reference layer.

The selective reception of the analyte on the test cantilever has the effect that a force acts on the test cantilever by the analyte, with the result that the test cantilever reacts sensitively to the analyte. Accordingly, the other substances of the sample which are not the analyte contribute only to a background noise in the form of a background bending on the test cantilever. The force on the test cantilever increases, for example, the faster the greater the concentration of the analyte in the sample or the faster the surface of the cantilever is covered with the analyte. A maximum force possible for the respective design is achieved with a complete covering of the cantilever.

The selective non-reception of the analyte on the reference cantilever, by contrast, has the effect that no force acts on the reference cantilever by the analyte, with the result that only the substances which are not the analyte contribute to a background noise in the form of a background bending of the reference cantilever.

This acting force can bring about a deformation in the case of the deformable part of the test cantilever, while the deformable part of the reference cantilever is not bent. The basis for the deflection of the cantilever is the change in the surface tension as a result of the interaction with the analyte. The change in the surface tension leads to an expansion or compression of the upper (or lower) surface of the cantilever. The different expansion or compression on the upper side and underside brings about an internal force or material stress in the material, which internal force or material stress leads to the deformation.

Reference cantilevers according to the prior art have only no receptor layer which reacts sensitively to the analyte. Effects such as turbulence in the sample and the thermal drift of the sensor system can indeed be determined thereby. However, in the case of such a reference cantilever, the analyte can bind to the reference layer of the reference cantilever for example by non-specific binding.

However, the analyte itself contributes to the background noise thereby. Therefore, in the case of a sensor according to the prior art, reference measurements in a reference sample, that is to say a sample without analyte, are necessary. Only the effect of the non-specific binding of the substances which are not the analyte can be determined thereby.

In the case of the biosensor, the measuring method is drastically simplified by the selective non-reception of the analyte by the reference cantilever, since the reference cantilever is not sensitive to the analyte and therefore the analyte also does not contribute to the background noise. Only the substances which are not the analyte contribute here to the background noise of the reference cantilever. To a certain extent, the selective non-reception of the analyte on the reference cantilever can have the effect that the reference cantilever is exposed to the same turbulences, the same thermal drift and the same influence of all substances which are not the analyte, as in a reference sample. However, with the difference that the reference signal is determined directly in the sample liquid.

In particular, a reference cantilever with a reference layer and a test cantilever with a receptor layer bring about a significantly more specific analysis of the analyte than only a reference cantilever without a receptor layer, since both the reference layer and the receptor layer have a specific interaction or non-interaction with the analyte.

The construction of the sensor with reference cantilever and test cantilever has the advantage that two measurements can be carried out simultaneously in the sample, wherein the measurement of the reference cantilever can calibrate the measurement of the test cantilever. Environmental influences, for instance chemical, thermal, mechanical, electrical and fluidic interference influences, on the respective measurement can thereby be reduced, with the result that an occurrence of the analyte can be inferred from the comparison of the measurement on the test cantilever and on the reference cantilever.

These forces or material stresses, for example expansions or compressions, which act on the cantilever can finally be detected by the transducers, wherein differently strong stresses are detected by the transducers by differently strong expansions or compressions.

The test cantilever 2 and the reference cantilever 3 thus measure the occurrence of the analyte 90 of the sample 9, however, at different positions. Different properties of the sample can occur at different positions of the sample, such as, for example, temperature fluctuations or concentration gradients. These different environmental conditions can be measured with the passive transducers 200, 300.

The passive transducers 200, 300 are arranged on the base and preferably do not detect a measurement signal in the event of a deformation or change in the surface tension of the deformable part 22, 32 of the reference cantilever or test cantilever 2, 3. However, the base level of the measurement signal of the passive transducers 200, 300 can be influenced based on these different environmental conditions. By providing a respective comparison value for each measurement value of the active transducers 220, 230 via the passive transducers 200, 300, which comparison value considers the environmental conditions in isolation, the influence of the environmental conditions on the measurement signals of the active transducers 220, 320 can be determined and reduced or calculated out or isolated.

Accordingly, the occurrence of an analyte 90 in a sample 9 can be analyzed in isolation via the biosensor 1 by reducing and isolating the influence of interactions which are not to be assigned to the analyte 90 by a plurality of measurement points on the reference cantilever and test cantilever 3, 2. This enables a high measurement accuracy of the occurrence of the analyte 90 in the sample 9.

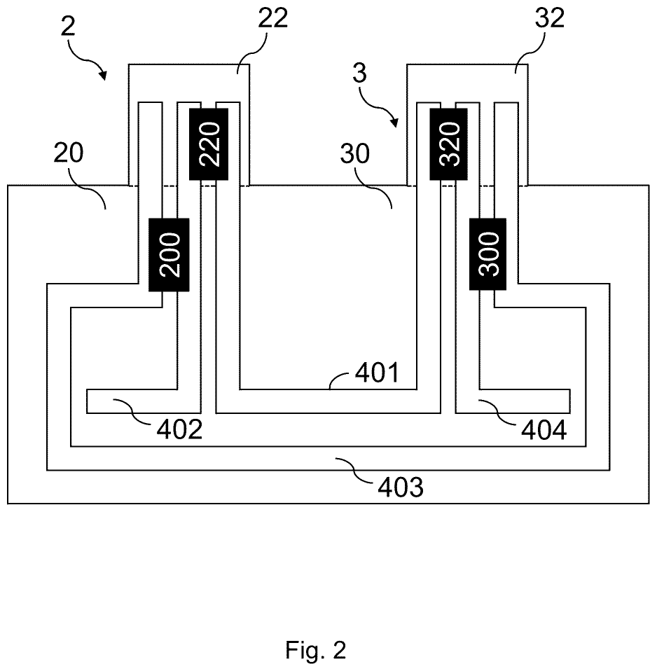

FIG. 2 shows a further schematic embodiment of a biosensor 1. The transducers 300, 320, 200, 220 are electrically connected via the electrodes 401, 402, 403, 404. In particular, the active test transducer 220 is connected to the active reference transducer 320 via the electrode 401. Furthermore, the passive test transducer 200 is connected to the passive reference transducer 300 via the electrode 403. The active test transducer 220 is additionally connected to the passive test transducer 200 via the electrode 402, whereas the active reference transducer 320 is connected to the passive reference transducer 300 via the electrode 404.

This results in a total of four electrodes via which the transducers 200, 220, 300, 320 are electrically connected to one another. In this case, electrical connection can be achieved, in particular, by the transducers being applied to the electrodes, with the result that a conductive connection arises.

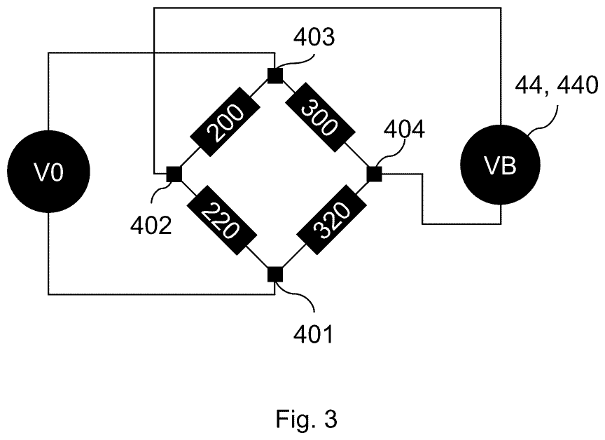

FIG. 3 shows a possibility of connecting the electrodes. The electrodes which electrically connect the transducers 200, 220, 300, 320 are overall of mirror-symmetrical construction. Currents run through the electrodes or voltages are present, with the result that, with an asymmetrical design of these electrodes, an asymmetrical crosstalk of electrical signals to the other electrodes could occur. As a result of this mutual influencing, a stray signal can be generated between the electrodes, but this can be avoided by the symmetrical design. Consequently, voltages drop at the electrical resistances of the transducers 200, 220, 300, 320, such as, for example, a single-ended voltage at the passive test transducer U_{test passive (200)}, and a single-ended voltage at the passive reference transducer U_{reference passive (300)}.

Here, the transducers 200, 220, 300, 320 are electrically connected in a so-called resistance bridge. In the resistance bridge, an excitation voltage V 0, for example DC voltage or AC voltage, is applied between the electrodes 403, 401. The passive and active transducers act as voltage dividers between these electrodes, on account of their electrical resistances. resistance bridge in the form shown has the advantage that no voltage is built up between the electrodes 402, 404 if the ratio of the electrical resistances of the passive transducer 200 to the active transducer 220 of the test cantilever 2 is equal to the ratio of the electrical resistances of the passive transducer 300 to the active transducer 320 of the reference cantilever 3. Therefore, in particular, the deviation of an electrical resistance is sufficient to change the electrical resistance ratios and thus to build up a voltage between the electrodes 402, 404.

If the reference cantilever 3 and the test cantilever 2 interact with the sample 9 and the analyte 90, both deformable parts 22, 32 experience, for example, a change in the surface tension which is greater, for example, for the deformable part 22 of the test cantilever 2 than for the deformable part 32 of the reference cantilever 3. Consequently, the electrical resistance of the active test transducer 220 of the deformable part 22 of the test cantilever 2 will vary to a greater extent than for the active reference transducer 320 of the deformable part 32 of the reference cantilever 3. If the electrical resistances of the passive transducers 200, 300 do not change or at least change equally, a change in the electrical resistance ratios results from the deformation of the deformable part 22 of the test cantilever 2 on account of the interaction with the analyte 90 of the sample 9 which interacts specifically with the reference layer 24 of the test cantilever 2. In the case of such an interaction, a voltage is accordingly built up between the electrodes 402, 404, with the result that a change in the surface tension in the case of the active test transducer 220 relative to the active reference transducer 320 can be indicated as bridge transverse voltage VB. Preferably, the bridge transverse voltage VB scales with the occurrence of the analyte 90 in the sample 9, with the result that a quantitative evaluation of the measurement signal is enabled.

The bridge transverse voltage VB corresponds to the difference between the single-ended voltage at the passive test transducer U_{test passive (200)} and the single-ended voltage at the passive reference transducer U_{reference passive (300)}.

A bridge transverse voltage detector 44 can indicate or forward the bridge transverse voltage VB to the outside, with the result that it becomes visible to the user of the biosensor 1 that a bridge transverse voltage VB is present. In particular, such a bridge transverse voltage detector 44 can also be given by an AD converter 440, wherein the AD converter 440 converts the bridge transverse voltage VB into a digital signal which can be forwarded to the external measurement apparatus.

In particular, the AD converter 440 can be operated in two different measurement modes.

The first measurement mode is the differential measurement mode in which the bridge transverse voltage VB is measured and therefore a relative measurement value for the deformation of the two reference cantilevers and test cantilevers 3, 2 is generated. In this differential measurement mode, the measurement signal of all the transducers 200, 220, 300, 320 is to some extent taken into consideration, with the result that the output signal of the AD converter 440 is a measurement signal which is cleaned of environmental influences and by means of which the relative deformation of the deformable parts 32, 22 and therefore the occurrence of an analyte 90 can be inferred.

The second measurement mode is the so-called absolute measurement mode. In the absolute measurement mode, the bridge transverse voltage VB is not detected, but rather the signals at the electrodes 402 and 404 are tapped off in isolation from one another, with the result that a statement about the respective deflections of the deformable parts 32, 22 can be made. This information is denied to the user in the differential measurement mode in which the bridge transverse voltage VB is measured.

The biosensors actually produced can have a certain scattering width with regard to the respectively applied structures and therefore in particular also with regard to the properties of the transducers, in particular of the active and the passive test transducer 220, 200 and of the active and passive reference transducer 320, 300. These differences in the properties, for example in the electrical resistances of the transducers 200, 220, 300, 320, are production-related and accordingly cannot be completely ruled out.

However, it is of great importance for the user of a biosensor 1 that the respective analysis results are consistent and reliable or the user receives an indication of the probability or the certainty with which the respective analysis results are affected.

In other words, the user wishes to know whether the test result obtained is reliable or not. However, the expected degree of reliability can vary depending on the field of application. In a virus test for humans, a very high level of reliability can be required in order, for example, to reliably order or cancel quarantine measures. In a lactate measurement for a leisure sportsman, the expected reliability can also be lower.

In order to quantify the reliability of tests, for example, the parameters of the sensitivity and of the specificity are known, by means of which a test can be classified. For example, in a test for the presence of a specific virus, these performance parameters can be specified in order to describe the reliability of the test.

The sensitivity of a test specifies which proportion of the tested persons is reliably recognized as sick (“positive”) by the test. The sensitivity is correspondingly defined as the number of persons tested as sick by the test divided by the number of persons actually sick in the group of persons tested with the test. In the case of a high sensitivity of the test, hardly a sick person is incorrectly evaluated as healthy, so that in a test with a high sensitivity a sick person is also recognized as sick with a high probability.

The specificity of a test specifies which proportion of the tested persons is reliably recognized as healthy (“negative”) by the test. The specificity is correspondingly defined as the number of persons tested as healthy by the test divided by the number of persons actually healthy in the group of persons tested with the test. In the case of a high specificity of the test, hardly a healthy person is incorrectly evaluated as sick, so that in a test with a high specificity a healthy person is also recognized as healthy with a high probability.

A method is described below, by means of which the biosensors described above can be individualized correspondingly for measuring a specific analyte and the measuring method is then configured correspondingly such that the test results always enable a measuring result with a high sensitivity and a high specificity despite a possible range of fluctuation during the production of the biosensors.

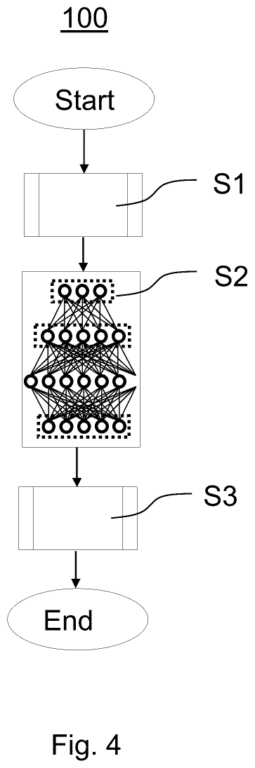

FIG. 4 shows a schematic flowchart of an exemplary embodiment for a method 100 for individualizing an individual, produced biosensor 1 for measuring a specific analyte 90. The individualization of the individual, produced biosensor 1 is to be carried out to the effect that the individual biosensor 1 can be used for a specific application scenario after successful individualization by the user. In the corresponding application scenario, it is then provided that the biosensor 1 carries out the corresponding analysis such that the requirements on the reliability of the test, for example on the sensitivity and the specificity, expected by the user for the specific application scenario can always be achieved, or the test is rejected. In other words, a reliable test result can always be achieved in this way.

In the method 100, at least one quality parameter of the individual biosensor 1, the individualization of which is to be achieved, is determined in a first step S1. The at least one quality parameter can be used as an indicator for the performance of the biosensor 1. At least the at least one quality parameter serves in the further method 100 for individualizing the biosensor.

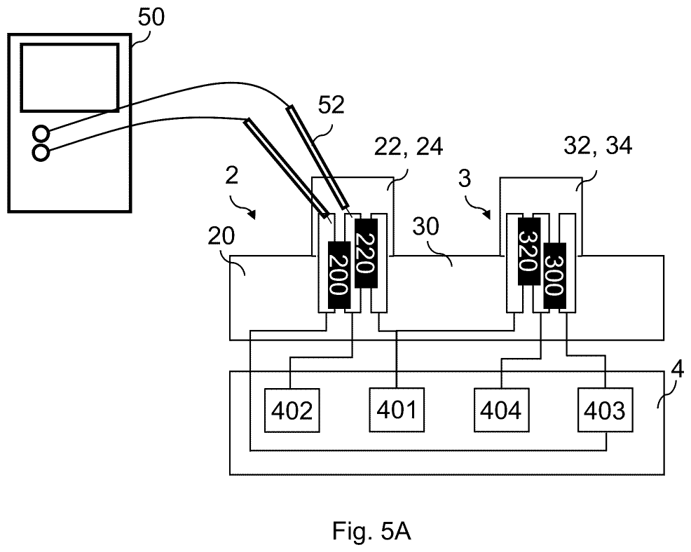

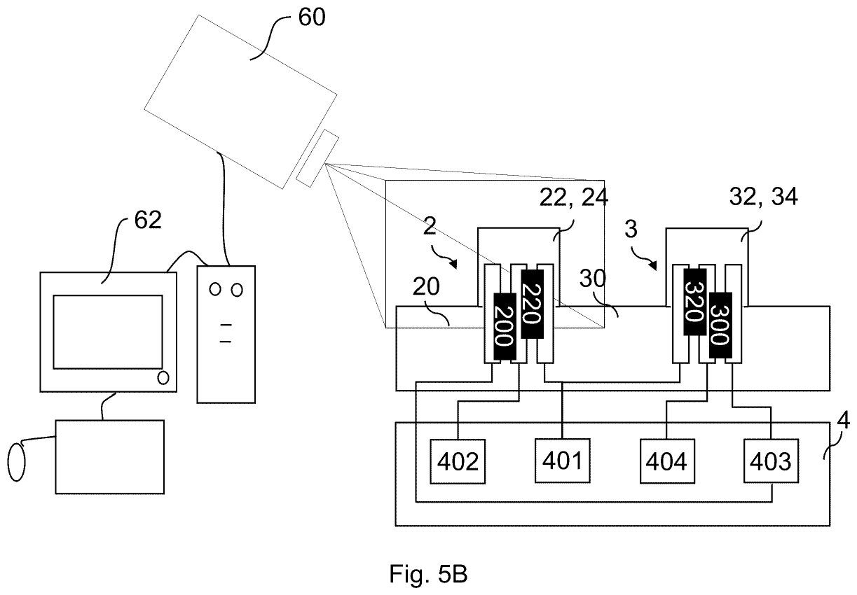

The at least one quality parameter of the individual biosensor 1 results for example from the production data of the biosensor 1 which were recorded during the production of the biosensor 1 or from a measurement. In this case, the quality parameters can be determined directly either by the production data or the measurement data, or can be determined from the production data or the measurement data. An exemplary determination of measurement data or a determination of quality parameters from the measurement data or from production data is described below for example in FIGS. 5A and 5B. Reference is made to this at this point.

In a second step S2, the at least one performance parameter and a corresponding threshold value are then determined for the individual biosensor 1 based on the quality parameters provided. The threshold value preferably corresponds to a probability on the basis of which a correctness of a measurement result is assessed by a measurement with the biosensor. The correctness can assume the values correctly or invalidly. The corresponding threshold value is dependent on the performance parameters of the biosensor 1. For example, the sensitivity, specificity and/or the yield of the respective biosensor 1 for measuring a specific analyte 90 can be determined as performance parameters. The sensitivity or specificity specifies how reliably a medical diagnostic method, such as for example the measurement of a specific analyte 90 in a user, recognizes whether one is sick or not sick. The yield in turn specifies to what extent a medical diagnostic method, such as for example the measurement of a specific analyte in a user, delivers a correct result.

The performance parameters are determined by means of a trained first artificial intelligence, wherein the trained first artificial intelligence determines the performance parameters and threshold values thereof from the quality parameters of the respective individual biosensor 1. In this case, the trained first artificial intelligence outputs a list of performance parameters and associated threshold values after the input of the at least one quality parameter.

For example, the determination in step S2 results in a list, which is individual for the respective biosensor 1, of tuples with performance parameters and respectively associated threshold values based on the at least one quality parameter of the respective biosensor 1. Each tuple from the list of tuples comprises a value and an associated threshold value for each performance parameter. With a suitable choice of the performance parameters, the tuple allows an evaluation of the correctness of a measurement of the biosensor 1 based on the associated threshold value.

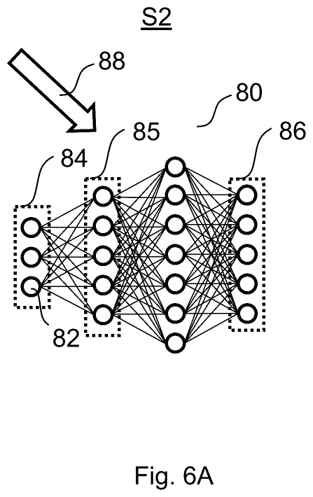

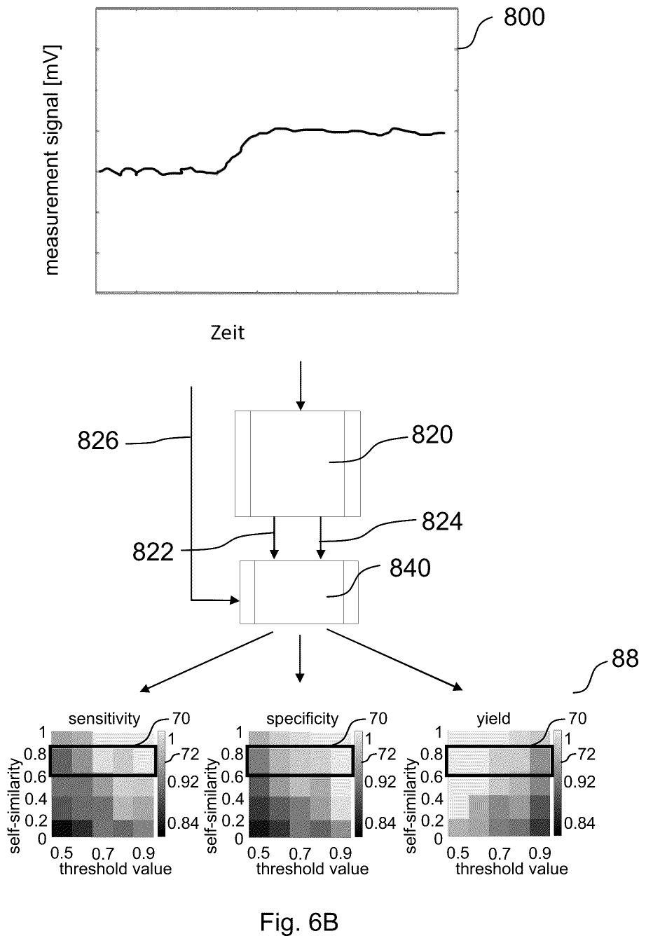

As shown in the exemplary embodiment 6A, a neural network 80 can be used as a trained first artificial intelligence for determining performance parameters. The training of the first artificial intelligence is described below subsequently to FIG. 6A. Reference is made to this at this point.

The training of the first artificial intelligence specifies this for the task in step S2. Thus, the first artificial intelligence used in step S2 can determine lists for one or more performance parameters with the respective threshold values for each, or a specific, analyte. For example, the sensitivity, specificity and/or the yield of the respective biosensor 1 for measuring a specific analyte 90 can be determined by the trained first artificial intelligence as performance parameters.

In a third step S3, an individualization of the individual biosensor 1 is then carried out based on the determined performance parameters and the associated threshold value to the effect that the performance parameters and their threshold values are analyzed for different application scenarios and in particular analytes 90 to the effect whether the individual biosensor 1 is suitable for a specific application scenario, or more for another application scenario.

For example, the biosensor 1 is to be used in a first application scenario for detecting a virus for containing a pandemic. In this case, the analyte 90 is clearly specified, since this is the respective virus which occurs in the sample liquid of the sample 9 to be tested. Furthermore, in order to be applicable correspondingly for this case, a high sensitivity and a high specificity of the test must be achieved. For this first application scenario, a requirement profile correspondingly results to the effect that the analyte 90 must be determined in the form of the virus with a high specificity and at the same time a high sensitivity, and taking account of the yield. A high sensitivity and a high specificity are of great importance here, inter alia, in order, on the one hand, to be able to contain the pandemic, but, on the other hand, also to be able to reliably implement official measures for individual restriction of freedom, such as quarantine measures. The test usually has both a specificity and a sensitivity of 100% in each case here. A high yield is important for reasons of resource conservation and user satisfaction in order to provide sufficient tests whose correctness of the measurement of the specific analyte are not invalid, for example.

In a second exemplary application scenario, the determination of the lactate content in the blood of a leisure sportsman may be required, for example. Here too, the analyte 90 is clearly specified, namely lactate in the sportsman's blood. The requirements on the sensitivity and specificity of the corresponding biosensor are, however, not so high in this case, for example, since the leisure sportsman does require the data for individual training control, but so that no substantial restrictions, for example of personal freedom, are associated with quarantine measures. Correspondingly, in this second application scenario, the analyte would be clear, namely lactate in the blood, but the requirements on specificity and sensitivity are significantly lower.

Specifically, in step S3, for example, based on the performance parameters determined in step S2 and the associated threshold values, it can be determined whether an individual biosensor 1 is suitable, for example, for a virus test for containing a pandemic, which requires a high sensitivity and a high specificity, or whether the individual biosensor 1 is more suitable for an application scenario in which a high specificity and high sensitivity are not required.

If a list of tuples is present, a tuple can be selected from the list of tuples. The selection of the tuple can preferably take place on the basis of the values for the performance parameters in the list of tuples taking account of the application scenario, in order to undertake the individualization of the biosensor in this way.

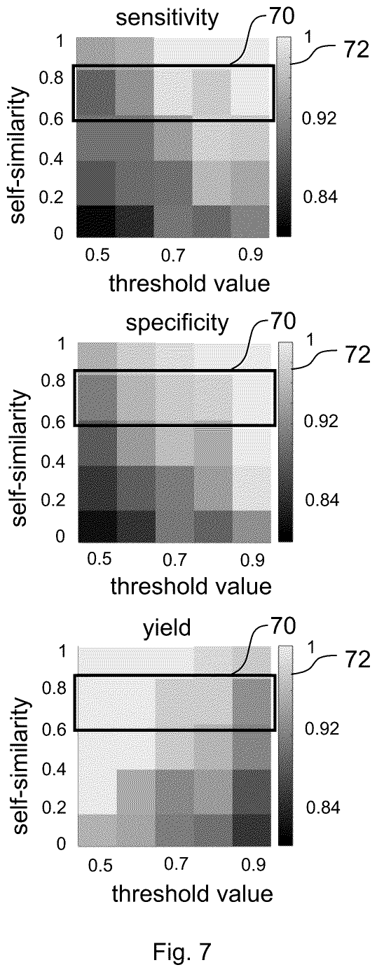

The threshold value in the selected tuple can be used later in the measurement of the specific analyte by the biosensor for assessing the correctness of the measurement. The threshold value depends on the performance parameters. This will also be discussed below in the description of FIG. 7. Reference is also made to this. Correspondingly, however, the tuple and in particular the threshold value for the respective performance parameter can also advantageously be stored with the biosensor 1.

After completion of the individualization, the method 100 ends and the individual, now individualized biosensor can be marked and used corresponding to the respective defined application scenario.

For this purpose, both the application scenario and the associated threshold value can be stored in the respective individual, now individualized biosensor.

Of course, with a corresponding evaluation of the individual biosensor, a plurality of different application scenarios with the associated threshold values can also be determined and stored, which are then selectably stored in the biosensor.

The storage of the data, i.e. the corresponding application scenario and the performance parameters and threshold values related thereto, can be stored in a non-volatile memory in the biosensor, for example an EEPROM. The individualization of the respective individual biosensor can be concluded by the storage of the data.

The individualization of the biosensor for measuring a specific analyte can also take place in step S3 using the trained first artificial intelligence from step S2 including a specific application scenario. The trained first artificial intelligence consequently determines the performance parameter values and the corresponding threshold value for the biosensor 1 based on the at least one quality parameter including the specific application scenario. The trained first artificial intelligence selects, for example, a tuple from a list of tuples based on the specific application scenario. The performance parameter values and the associated threshold value in the selected tupel are based on the specific application scenario and the trained first artificial intelligence performs an individualization of the biosensor 1. The specific application scenario can be predefined, for example, by a user, or determined on the basis of current inventory and demands and provided to the trained first artificial intelligence.

If no suitable tuple can be selected by the trained first artificial intelligence because, for example, no performance parameter values for the biosensor 1 exist for the specific application scenario, the first artificial intelligence can output an error message. In this case, the trained artificial intelligence can also independently select another specific application scenario, for example, on the basis of the current inventory and/or demand. The first artificial intelligence acquires the specific application scenario in order to define a performance parameter with an associated threshold value for the biosensor 1. Accordingly, the training of the first artificial intelligence takes the specific application scenario into consideration. The taking into account of the specific application scenario in the training of the first artificial intelligence is explained in more detail at the end of FIG. 6B. Reference is made to this at this point.

By way of example, the determination of at least one quality parameter by the measurement of measurement values or the evaluation of production data is shown below in FIGS. 5A and 5B.

FIG. 5A schematically shows an exemplary embodiment for determining a property of the biosensor 1 with reference to an exemplary embodiment of a biosensor 1 comprising a test cantilever 2 and a reference cantilever 3 with deformable parts 22, 32. By means of a measuring device, here illustrated by way of example in the form of a multimeter 50, the voltages at the electrical resistances of the transducers 300, 320, 200, 220, such as, for example, the single-ended voltage at the passive test transducer U_{test passive (200)} and the single-ended voltage at the passive reference transducer U_{reference passive (300)}, are measured. The transducers 300, 320, 200, 220 are electrically connected via the electrodes 401, 402, 403, 404. For this purpose, measurement tips 52 of the multimeter 50 are correspondingly positioned at the electrodes of the biosensor 1. The measured value of the voltage at the electrical resistance is indicated in the exemplary embodiment on a display of the multimeter 50. The multimeter 50 can also be used for measuring other properties of the biosensor 1, such as, for example, electrical resistances, capacitances or impedances. For example, the multimeter can be used for measuring the electrical resistances of the transducers 300, 320, 200, 220. The multimeter 50 can also be part of a measuring apparatus, in particular during or after the production process of the biosensor, such that the measurement of the at least one property can proceed in an automated manner.

FIG. 5B shows a further exemplary embodiment for determining the at least one property of the subcomponents or all subcomponents of the biosensor 1 with a camera system 60. The camera system 60 can record an image section of the biosensor 1 by an imaging method, create a spectrum of the image section, evaluate a time-resolved analysis, a spectrally filtered excitation and spectrally filtered detection of signals by means of a camera and, for example, by means of a computer 62. The evaluation can take place in all production steps and possibly include the use of electrons, light, ions and/or near-field analyses.

The evaluation of the image section can result, for example, in a determination of the electrical resistances of the transducers 300, 320, 200, 220 as a property of the biosensor 1. The transducers 300, 320, 200, 220 are electrically connected via the electrodes 401, 402, 403, 404. The evaluation of the image section can also result in the occupation density and homogeneity of a coating of a test cantilever 2 and of a reference cantilever 3 as a property of the biosensor 1. The evaluation of the image section can also be the individual, finely resolved, geometric configuration of the transducer, of the electrodes or of the transducer and can also contain spectroscopic information.

At least one quality parameter can then be determined from the previously determined properties of the biosensor.

For example, a proportion and the distribution thereof of the functionalized surface of the biosensor 1 can be determined as a quality parameter.

The self-similarity of the electrical resistances in the resistance bridge of the biosensor 1 can also be determined as a quality parameter from the previously determined single-ended voltages at the electrical resistances of the passive test transducer U_{test passive (200)}, of the passive reference transducer U_{reference passive (300)} and of the excitation voltage V0.

The self-similarity of the biosensor 1 results here in a first consideration, for example on the basis of the following calculation rule with inclusion of the excitation voltage V0 at the resistance bridge, the single-ended voltage at the passive test transducer U_{test passive (200)} and the single-ended voltage at the passive reference transducer U_{reference passive (300)} as follows:

$$Self - similarity = min ( 1 - \vert {U}_{test passive ( 200 )} / ( V 0 / 2 ) - 1 \vert ,$$

$$1 - \vert {U}_{reference passive ( 300 )} / ( V 0 / 2 ) - 1 \vert ) ,$$

wherein min (,) outputs the smallest value of the arguments. The single-ended voltages at the electrical resistances of the passive test transducer U_{test passive (200)} and of the passive reference transducer U_{reference passive (300)} can be determined here from the electrical resistances R_{reference passive (200)}, R_{reference active (220)}, R_{test passive (300)}, R_{test active (320)} of the transducers 200, 220, 300, 320 in the resistance bridge and the excitation voltage V 0 at the resistance bridge as follows:

$${U}_{test passive ( 200 )} = V 0 * {R}_{test passive ( 200 )} / ( {R}_{test passive ( 200 )} + {R}_{test active ( 220 )} ) ,$$

$${U}_{r eference passive ( 300 )} = V 0 * {R}_{r eference passive ( 300 )} / ( {R}_{r eference passive ( 300 )} + {R}_{reference active ( 320 )} ) .$$

This results in the self-similarity depending on the electrical resistances R_{reference passive (200)}, R_{reference active (220)}, R_{test passive (300)}, R_{test active (320)} of the transducers 200, 220, 300, 320 as follows:

$$Self - similarity = min ( 1 - \vert ( {R}_{test passive ( 200 )} - {R}_{test active ( 220 )} ) / ( {R}_{test passive ( 200 )} + {R}_{test active ( 220 )} ) \vert ,$$

$$1 - \vert ( {R}_{reference passive ( 300 )} - {R}_{reference active ( 320 )} ) / ( {R}_{reference passive ( 300 )} + {R}_{reference active ( 320 )} ) \vert ) ,$$

The self-similarity can possibly also be determined including the bridge transverse voltage VB.

As already described above, the trained first artificial intelligence used in step S2 can be a trained neural network 80. In FIG. 6A, the trained neural network 80 from step S2 is shown once again as an exemplary embodiment as a possibility for a first artificial intelligence. The trained first artificial intelligence, i.e., for example, the neural network 80 from FIG. 6A, is here trained with specific training data in order to enable the determination of the performance parameters from the quality parameters for the respective biosensor 1 accordingly, wherein the generation and application of the training data on the basis of which the first artificial intelligence can be trained for the use in step S2 will also be discussed below.

In step S2, the neural network 80 shown in FIG. 6A determines the performance parameters of the biosensor 1 and associated threshold values for an analyte 90 on the basis of a feature vector. The feature vector results from the at least one determined quality parameter of the biosensor 1.

The feature vector can be obtained, for example, from a data preparation of the at least one quality parameter. The data preparation can contain, for example, a rescaling of the at least one quality parameter. The data preparation can also include an error analysis of the at least one quality parameter, wherein the error analysis ends with a generation of the feature vector. The data preparation can here include the removal of unnecessary quality parameters. A main component analysis or another method for data reduction, or data selection, can also be used in the data preparation in order to obtain the feature vector which comprises as much information as possible of the underlying at least one quality parameter. The data preparation can also include a storage of the at least one quality parameter and/or the feature vector in a database or in a memory, such as, for example, an EEPROM, of the biosensor 1. The feature vector can also correspond to the at least one quality parameter.

The neural network 80 shown by way of example in the exemplary embodiment in FIG. 6A has four layers. A neural network 80 can also have a topology with, for example, more or fewer layers, and also more or fewer neurons in the respective layers.

The feature vector based on the quality parameters is transferred as an input value to the trained neural network 80. For example, the feature vector can have n components \vec{x}=(x₁, . . . , xₙ), wherein the neural network 80 accordingly has n neurons 82 in a first layer 84. The neural network 80 in the exemplary embodiment in FIG. 6A has three neurons 82 in its first layer 84. Therefore, feature vectors with three components are transferred to the neural network 80. A neuron 82 at the i-th position in the second layer 85 of the neural network 80 outputs a value as follows from σᵢ(\vec{w}ᵢ^{T} \vec{x}+bᵢ), wherein \vec{w}ᵢ=(w₁, . . . , wₙ) is a vector with weights and bᵢ is a bias value. The activation function can be, for example, a ReLU or sigmoid function.

The neural network 80 outputs performance parameters and their associated threshold values as output values at its last layer 86. In the exemplary embodiment in FIG. 6A, the neural network 80 has five neurons 82 in its last layer 86, wherein the five neurons 82 output a performance parameter list 70. A performance parameter list can comprise, for example, a list of tuples, wherein each tuple from the list of tuples comprises a value and an associated threshold value for each performance parameter.

The five neurons 82 can output, for example, five values for the performance parameter specificity in the form of a performance parameter list with respectively associated threshold value. For example, the specificity of the biosensor 1 is selected from the performance parameter list with the five values for the specificity and their associated threshold values.

The trained neural network 80 in FIG. 6A is trained by training data 88 and is suitable for fulfilling the function described in S2.

FIG. 6B schematically illustrates how the training data 88 can be obtained.

With a plurality of already produced biosensors 1, a measurement is carried out under controlled conditions in each case. In other words, a biosensor 1 is used under the regular measurement conditions, that is to say the measurement conditions for which the respective biosensor 1 is provided to the user, and under regular environmental influences which are to be expected in a measurement, for measuring a known analyte concentration of the analyte 90 of interest. Equally, a plurality of measurements can also be carried out under controlled disturbances, in order to create a more global topography. The profile of the resulting time-resolved measurement is stored here for each biosensor 1 of the produced biosensors in combination with the information about the measured analyte 90—in particular the known analyte concentration and/or the presence or absence of the analyte 90 in the sample liquid of the sample 9 to be analyzed.

A time-resolved measurement curve 800 shown by way of example for a biosensor in FIG. 6B results here for each individual biosensor 1 from the plurality of already produced biosensors. The measurement correspondingly delivers a measurement curve 800, wherein the measurement curve results based on a voltage signal, which varies over the time profile, at the resistance bridge of the biosensor 1.

It is also known which analyte 90 was measured at which concentration and at which disturbance, so that both the measurement curve 800 which is individual for each biosensor 1 and the result to be expected are known here. In this way, training data for training a second artificial intelligence 820 can be provided.

In the second artificial intelligence 820, for example a classifier, the individual measurement result 822 is determined from the time-resolved measurement curve 800 of an individual measurement and a validity parameter 824 is output simultaneously.

For example, the second artificial intelligence 820 can be a classifier which classifies the measurement result into a positive or negative individual measurement result. For this purpose, the second artificial intelligence 820 can comprise, for example, a dynamic time warping (DTW for short). The second artificial intelligence 820 can also comprise a regression model. The regression model can output an analyte concentration as an individual measurement result.

The validity parameter 824 is compared with a predefined threshold value in order to indicate the correctness of the measurement result. The threshold value stored in the individualization of the biosensor together with the application scenario is possibly used as a predefined threshold value for the evaluation of the validity parameter in the evaluation of the measurement. The correctness can assume the values correctly or invalidly.

The present measurement result can be invalid, for example, if a time-resolved measurement curve 800 ends abruptly as a result of a premature termination of the associated measurement and an unknown profile of the measurement curve is correspondingly present. In this case, the second artificial intelligence 820 can indeed determine the individual measurement result for the measurement curve, but only with a very low validity. Based on the comparison of the associated validity parameter with the predefined threshold value, the associated measurement result is then possibly classified as invalid.

Simultaneously, before each measurement of a specific analyte with a biosensor 1, its quality parameter is also determined, for example the self-similarity of the electrical resistances in the resistance bridge.

In a sorting step 840, the determined data, consisting of the individual measurement result 822, the associated validity parameter 824, the associated quality parameter, for example the self-similarity 826, as well as the known result for each biosensor from the plurality of already produced biosensors, are statistically evaluated.

The statistical evaluation can comprise the following substeps. The sorting step 840 can comprise a filtering of the determined data at the beginning. During the filtering of the determined data, each associated validity parameter is compared with a defined threshold value in order to indicate the correctness of each individual measurement result. All individual measurement results whose correctness is correct, for example, are combined with the associated validity parameters, as well as their associated quality parameters and the results to be expected to form a filtered data set. The filtered data set is in turn divided into groups, wherein each group includes biosensors with the same quality parameters. A group can possibly also comprise all associated data from the filtered data set of which one or more quality parameters lie above one or more predefined limit values. For each of these groups, the performance parameters, such as, for example, sensitivity, specificity and/or yield, can now be determined. The filtering and subsequent determination of the performance parameters is repeated for a plurality of defined threshold values in order to generate the training data 88.

In FIG. 6B, the training data 88 are specified by way of example by a three-dimensional representation, for example for the sensitivity, wherein the values for the sensitivity are indicated based on a grayscale coding 72. The three-dimensional representation has plotted on the y-axis as quality parameter the self-similarity and on the x-axis the threshold value.

The training data 88 resulting therefrom, which, if they are applied to the trained first artificial intelligence, such as, for example, to the neural network 80 in FIG. 6A, as training data, lead to the fact that, when a quality parameter for a biosensor 1, for example the self-similarity, is input, a list of performance parameters 70, for example comprising sensitivity, specificity and/or yield, can be determined with the associated threshold values for the biosensor for the specific analyte 90.

The training data can also still be marked according to the specific application scenarios. Thus, for example, it can be stored in the training data that performance parameters within a region are suitable for a specific application, for example for lactate measurement or for the measurement of a specific virus. If the training data are marked according to the specific application scenarios, the corresponding training data can be used for training a first artificial intelligence, which comprises the individualization of the biosensor for measuring a specific analyte in step S3, as shown, for example, in FIG. 1, including a specific application scenario for the biosensor 1.

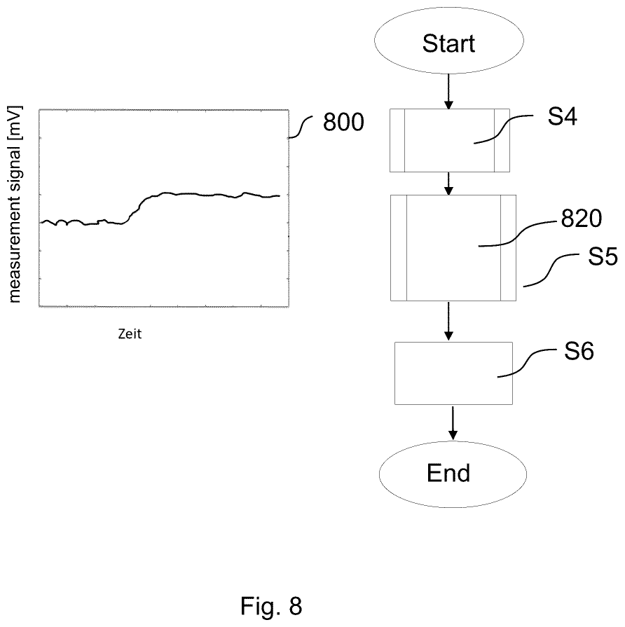

The second artificial intelligence 820 can also be used for assessing a measurement at a user (see in this regard FIG. 8).

The second artificial intelligence 820 is trained for the output of the individual measurement result and validity parameter. For training, stored measurement curves of the plurality of already produced biosensors can be used in combination with the information about the measured analyte—in particular the known analyte concentration and/or the presence or absence of the analyte in the sample liquid to be analyzed. These data are stored in a training data record.

If the second artificial intelligence 820 is, for example, a classifier, the classifier can be based on a nearest neighbor classification method. The classifier determines the measurement result, for example, for an unknown time-resolved measurement curve 800 of a biosensor on the basis of a comparison of the unknown measurement curve with the measurement curves in the training data set.

For the case of equally long and undistorted measurement curves, the comparison can take place, for example, by calculating in each case a standard between the still unknown measurement curve of the biosensor and all measurement curves in the training data set. The absolute values are then ranked in ascending order. The measurement result of the unknown measurement curve of the biosensor can result from a calculation rule, for example averaging, of a predefined number of measurement results for measurement curves from the training data set with the smallest absolute values. The validity parameter is calculated in this case, for example, on the basis of the highest absolute value for the measurement curve in the training data set, the measurement result of which is used in the determination of the measurement result of the still unknown measurement curve.

The second artificial intelligence 820 can be adapted to comprise a dynamic time warping (DTW for short). DTW is based on a method which allows a comparison of two time series, such as, for example, two time-resolved measurement curves 800, wherein the two time series do not have to match perfectly, but can, for example, be temporally shifted or distorted. The DTW enables, for example, two time series of different length to be compared with one another. Consequently, the comparison of two time-resolved measurement curves 800 which do not run over the same time duration can also be carried out by means of DTW.

If the second artificial intelligence 820 comprises, for example, a regression model, the regression model is trained by minimizing a cost function. The validity parameter can result from a calculation including the measurement result and the training data set.

FIG. 7 once again shows the training data 88 in three representations comprising sensitivity, specificity and yield.

The values for the performance parameters sensitivity, specificity and yield are illustrated on the basis of a grayscale bar 72. The self-similarity in the three representations lies between 0 and 1, and the threshold value lies between 0.5 and 0.9.

It can be recognized on the basis of the three representations that a systematic relationship is present for the sensitivity, specificity and the yield in each case depending on the self-similarity and the threshold value, and there is a relationship between the performance parameters sensitivity, specificity and yield among one another for a specific self-similarity and a predefined threshold value. The threshold value thus depends on the performance parameters. The threshold value also depends on the self-similarity.

The systematic relationship is manifested in the fact that a greater sensitivity and specificity tends to result with a specific self-similarity and with an increasing threshold value, even though the yield tends to decrease.

By including the predefined threshold value, the sensitivity, specificity and yield for the biosensor 1 can then be defined during the measurement based on the respective performance parameter lists 70. For the specific self-similarity of the biosensor 1, a matching between the sensitivity and the specificity, and the yield of the biosensor 1 can thus correspondingly be carried out by defining the threshold value.

FIG. 8 shows an exemplary embodiment for evaluating a measurement of a biosensor 1 on the basis of the individualization of the biosensor 1 with reference to a flowchart.

For this purpose, in the method 100, as already described above, the individualized biosensor for the application scenario for which it was individualized is used for measuring the corresponding specific analyte.

In step S4, the measurement is then carried out with the correspondingly individualized biosensor under the predefined measurement conditions. This results from the measurement of the time-resolved measurement curve 800, which correspondingly shows the profile of the signal voltage at the resistance bridge for the reference cantilever 3 and the test cantilever 2 in a time-resolved manner.

The specific analyte 90 can be measured using an apparatus. The apparatus also comprises, inter alia, the sample 9 to be measured with the specific analyte 90. An apparatus may possibly also comprise a plurality of technical components. The technical components may be, for example, one or more mobile devices. The apparatus may also include one or more programs. The one or more programs may be stored and/or run on the technical components, for example. The programs can serve for an evaluation of the measurement of the specific analyte including the individualized biosensor. The evaluation can be triggered, for example, by an electrical signal from the biosensor 1 to the apparatus. An evaluation of the measurement on the basis of data from the measurement of the analyte can also take place on a server. The data of the measurement, and also the data of the biosensors themselves, such as, for example, their at least one quality parameter, application scenarios, performance parameters and/or threshold value, can also be stored and processed on a cloud.

The resulting measurement curve 800 is supplied in step S5 to the second artificial intelligence 820, which outputs an individual measurement result and a validity parameter for the measurement result to be correct.

Depending on the validity parameter, the final measurement result is then output to the user in step S6 to the effect that, in a case in which the validity parameter falls below a predefined threshold value, the measurement result is classified as invalid. In the cases in which the validity parameter, however, lies above the predefined threshold value, a measurement result can be output which is positive or negative with a corresponding probability which corresponds at least to the validity parameter. The threshold value stored in the individualization of the biosensor 1 together with the application scenario is possibly used as a predefined threshold value for the evaluation of the validity parameter in the evaluation of the measurement. The threshold value from the individualization of the biosensor 1 preferably also results inter alia from the application scenario of the biosensor 1.

The comparison between validity parameter and the predefined threshold value can also take place in step S5 by the second artificial intelligence. Step S6 consequently comprises an output of the classification of the measurement result, the measurement result itself and/or the corresponding probability which corresponds at least to the validity parameter.

It can thus be achieved that the measurement result can always be designed absolutely reliably depending on the predefined application scenario by the individualization of the biosensor, on the one hand, and the evaluation of the measurement result on the basis of its validity parameter for the corresponding performance parameters, on the other hand. In an extreme case, it can be achieved in this way that, for example, both a specificity and a sensitivity of 100% in each case can be achieved for a virus test.

If it has thus been defined, for example, in the individualization of the biosensor for a specific application scenario that a specificity of 98% and a validity of 59% are required with a threshold value of 0.60, this threshold value must be used for evaluating the measurement for this individual biosensor in order to achieve the required performance parameters.

In other words, if the measurement carried out in the evaluation of the measurement curve in the artificial intelligence 820 in step S5 results in a validity parameter which lies below 0.95, the corresponding measurement is rejected as invalid. It is therefore always ensured that the parameters defined for the predefined application scenario are strictly adhered to by the measurement, or else the measurement is rejected as invalid.

Irregularities in a measurement can lead to an unknown or irregular profile of the measurement curve. By means of the training of the second artificial intelligence 820, a precise measurement result can nevertheless be determined for weak irregularities. A weak irregularity is present, for example, if the measurement is terminated shortly before completion, or weak disturbances, such as for example low temperature fluctuations, occur in the measurement. By means of repeated and adapted training of the second artificial intelligence 820, such weak irregularities in the measurement can be compensated for. Strong irregularities can arise, for example, as a result of a significantly early termination of the measurement, wherein, in the case of the termination, the biosensor 1 is separated from the specific analyte 90 significantly before the completion of the measurement. In the case of such an unknown or irregular profile of the measurement curve, the second artificial intelligence 820 will indeed likewise come to a result, but the validity parameter associated with the result will be very low. In other words, an irregular measurement will always lead to the evaluation of the validity parameter in relation to the threshold in step S6 leading to the respective measurement being rejected as invalid. Consequently, the number of measurements rejected as invalid can be reduced by means of a repeated and adapted training of the second artificial intelligence 820.

Since the individualization of the respective biosensor for the method shown in FIG. 8 for determining the measurement result is merely advantageous but not indispensable, the exemplary embodiment can also be regarded as starting with the method 100 for individualizing a biosensor 1. However, the individualization of the biosensor is not absolutely necessary.

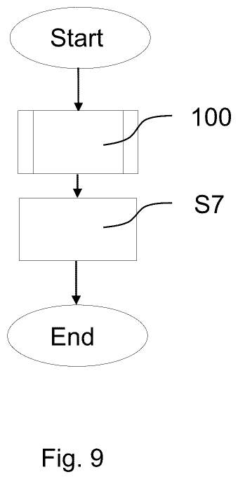

FIG. 9 shows an exemplary embodiment for running through no or at least one further production step S7 of a biosensor 1 on the basis of a method 100 for individualizing the biosensor with reference to a flowchart. As already described in FIG. 4, the successful individualization leads to a determination of an application scenario of the biosensor 1. Here, both the application scenario and the performance parameters associated with the application scenario with the respective threshold values can be stored in the biosensor 1. Based on the stored data, comprising the application scenario, the associated performance parameters with the respective threshold values, biosensors, for example in stock as inventory, can run through at least one further production step S7. Production steps can comprise a functionalization, for example. During the functionalization, a biosensor is correspondingly adapted on the basis of the stored data. The functionalization can comprise, inter alia, an application of a receptor layer 24 and/or reference layer 34. The production of a biosensor on the basis of its individualization can also be terminated, so that the biosensor runs through no further production steps. The production steps can take place during the production, during a repair and/or a check of the biosensor.

Thus, biosensors can also be flexibly adapted for their respective application scenario, for example, after longer storage times. Biosensors can also possibly run through a method 100 for individualizing the biosensor at any time and then be post-processed or their production terminated.

Insofar as applicable, all individual features which are illustrated in the exemplary embodiments can be combined with one another and/or interchanged without departing from the scope of the invention.

### REFERENCE NUMERAL LIST

100 Method
S1 determining at least one quality parameter
S2 determining at least one performance parameter and corresponding threshold value
S3 individualizing a biosensor
S4 carrying out a measurement
S5 classifying the measurement
S6 evaluating a measurement
S7 running through no or at least one further production step
1 biosensor
2 test cantilever
3 reference cantilever
4 electronics
9 sample
90 analyte
200 passive test transducer
220 active test transducer
300 passive reference transducer
320 active reference transducer
400, 401, 402, 403 electrodes
20 base
22 Deformable part
24 Receptor layer
30 Base
32 Deformable part
34 Reference layer
44 Bridge transverse voltage detector
440 AD converter
50 multimeter
52 measurement tips
60 camera system
62 computer
70 performance parameter list
72 grayscale coding
80 neural network
82 neuron
84 first layer
85 second layer
86 last layer
88 training data
800 measurement curve
820 second artificial intelligence
822 measurement result
824 validity parameter
826 quality parameter
V0 excitation voltage
VB bridge transverse voltage

1-16. (canceled)

17. A method for individualizing a biosensor for measuring a specific analyte, the method comprising:
determining at least one quality parameter of the biosensor;
determining at least one performance parameter and an associated threshold value of the biosensor based on the at least one quality parameter using a trained first artificial intelligence; and
individualizing the biosensor for measuring a specific analyte by evaluating at least one of the at least one performance parameter and the associated threshold value for an individual application scenario.

18. The method according to claim 17, wherein the individualizing of the biosensor for measuring the specific analyte takes place using a trained first artificial intelligence that includes a specific application scenario.

19. The method according to claim 17, further comprising determining the quality parameter based on a property of the biosensor, wherein at least one electrical resistance is determined as the property, preferably all electrical resistances of a resistance bridge of the biosensor.

20. The method according to claim 17, wherein the biosensor comprises at least one resistance bridge and at least one of the quality parameters is a self-similarity of the electrical resistances of the resistance bridge.

21. The method according to claim 17, wherein the at least one of the performance parameter of the biosensor comprises a sensitivity and/or a specificity and/or a yield.

22. The method according to claim 17, further comprising determining, by the trained first artificial intelligence, at least one of the at least one performance parameter and a threshold value based on a feature vector that is obtained from a data preparation of the at least one quality parameter.

23. The method according to claim 17, further comprising training the trained first artificial intelligence based on training data that is obtained by:
determining a time-resolved measurement curve of at least one specific analyte, as well as at least one sample without the specific analyte, each with a plurality of biosensors;
determining an individual measurement result and a validity parameter for each biosensor with a second artificial intelligence from the time-resolved measurement curve of the respective biosensor;
determining at least one quality parameter for each biosensor;
statistically evaluating the individual measurement results based on the associated validity parameters using threshold values, the associated quality parameters as well as the previously known result for each biosensor;
determining performance parameters from the statistical evaluation; and
using the quality parameters, performance parameters and threshold values as the training data.

24. The method according to claim 23, wherein the time-resolved measurement curve is recorded at at least one defined concentration of the analyte and/or that the time-resolved measurement curve is additionally recorded at a defined disturbance and/or that the time-resolved measurement curve is recorded at at least one defined measurement routine.

25. The method according to claim 17, further comprising training the trained first artificial intelligence based on training data that comprise a self-similarity of an electrical resistances of a resistance bridge of each biosensor as quality parameters.

26. The method according to claim 17, further comprising training the trained first artificial intelligence based on training data that comprise a sensitivity and/or a specificity and/or a yield of each biosensor as performance parameters.

27. The method according to claim 17, wherein the biosensor runs through no or at least one further production step based on a respective individualization, wherein the no or at least one further production step preferably comprises a storage of quality parameters and/or performance parameters and/or threshold values on the biosensor or a database and/or the at least one further production step comprises a functionalization of the biosensor, wherein the functionalization of the biosensor preferably comprises an application of a receptor layer and/or reference layer.

28. A method for generating training data for training a trained first artificial intelligence for use in the individualization of biosensors, the method comprising:
determining a time-resolved measurement curve of at least one specific analyte, as well as at least one sample without the specific analyte, each with a plurality of biosensors;
determining an individual measurement result and a validity parameter for each biosensor with a second artificial intelligence from the time-resolved measurement curve of the respective biosensor;
determining at least one quality parameter for each biosensor;
statistically evaluating the individual measurement results based on the associated validity parameters using threshold values, the associated quality parameters as well as the previously known result for each biosensor;
determining performance parameters from the statistical evaluation; and
using the quality parameters, performance parameters and threshold values as training data.

29. The method according to claim 28, wherein the time-resolved measurement curve is recorded at at least one defined concentration of the analyte and/or that the time-resolved measurement curve is additionally recorded at a defined disturbance and/or that the time-resolved measurement curve is recorded at at least one defined measurement routine.

30. The method according to claim 28, wherein the quality parameter determined for each biosensor comprises the self-similarity of the resistances in a resistance bridge of each biosensor.

31. A biosensor for measuring a specific analyte, the biosensor comprising:
at least one resistance bridge,
wherein the biosensor is individualized for a specific application scenario for measuring the specific analyte based on a quality parameter determined from the resistance bridge.

32. The biosensor according to claim 15, wherein one of the quality parameters is the self-similarity of the resistances of the resistance bridge.
